(19) United States
(12) Patent Application Publication
(10) Pub. No.: US 2025/0204809 A1
(43) Pub. Date: Jun. 26, 2025

(54) SYSTEM AND METHOD FOR BODY CORRECTION AT AN ELECTRONIC DEVICE

(71) Applicant: LENOVO (SINGAPORE) PTE. LTD., SINGAPORE (SG)

(72) Inventors: Inna Zolin, Cary, NC (US); Joshua Smith, Milton, FL (US); Matthew Fardig, Boonville, IN (US); Allen Richey, Frisco, TX (US)

(21) Appl. No.: 18/392,990

(22) Filed: Dec. 21, 2023

(51) Int. Cl. A61B 5/11; A61B 5/00; G06F 1/16; G08B 21/04

(52) U.S. Cl. CPC A61B 5/1116; A61B 5/6802; A61B 5/7405; A61B 5/742; A61B 5/7455; G06F 1/1684; G08B 21/0446; G06F 1/1624

(57) ABSTRACT

A correction system is provided that includes a sensor system comprising a sensor configured to obtain sensor data related to a position of a body part of a user in relation to an electronic device. The electronic device can include one or more processors configured to determine a position of the body part of the user based on the sensor data obtained from the sensor and determine whether the position of the body part is at a determined position related to ensuring physical health of the body part. The one or more processors are also configured to alert the user to vary the position of the body part when determining the body part is not at the determined position related to ensuring physical health of the body part.

### BACKGROUND

Embodiments of the present disclosure generally relate to electronic devices and more particularly to systems for detecting user positioning and correcting posture and hand positioning.

A wide variety of electronic devices are offered today. Examples of electronic devices include computers (e.g., laptop or tablet computers), mobile phones, cellular phones, iPhone, smart phones, tablets, iPads, iPods, personal digital assistants (PDAs), smart watches, or the like. As electronic devices become more compact, more functionality and also desired.

Individuals spend hours in from of personal computers, and other electronic devices that include some form of display and keyboard combination. Often when individuals use these electronic devices, such as laptops or smartphones, the user's head is not aligned with their shoulders. As a result, physical health issues can occur, including so called hunched-over laptop syndrome. In particular, the poor posture can lead to excessive pressure on the spine and protracting of the shoulders forward. Consequently, up to a fifty-pound (lbs.) or greater increase of the load resulting from the head onto the spine can occur.

In addition to posture related physical health issues, physical injuries can often occur to individuals' wrists, such as carpal tunnel syndrome. In particular, when a wrist is not correctly aligned with the hands during typing, extra strain and stress can be put onto the joints of the wrist. Over time this extra stress and strain can cause significant damage to the joints in the wrist.

In order to address the problems associated with poor posture, manual posture belts have been provided. These manual posture belts physically hold a person's core in a determined position. Alternatively, other devices have been created that attach to an individual's back to detect the position of the spine. Both solutions have drawbacks in that a user must store these devices that are typically very bulky. To this end, such devices are not conducive to traveling. In addition, there can be time and frustration with having to put these devices on, when often it is quicker and easier to simply not use the devices. In addition, these types of devices focus on supporting and detecting the spine below the neck, that often does not solve the mispositioning of an individual's head and shoulders.

To address the problem of addressing mispositioning of the wrist, wrist supports exist that limit the mobility of the wrist. Another solution includes utilization of a keyboard wrist pad that is placed in front of the keyboard. Problematically with both, similar to the posture equipment, this equipment must be stored, can be bulky, and difficult to maintain. In addition, often equipment is designed with a one size fits all modality when in fact one size does not fit all. To this end, no adjustments can be made to accommodate different sized individuals. Thus, a need exists for improving the positioning of an individual's body parts such as their head, shoulders, wrists, hands, etc. in relation to a keyboard or display of an electronic device to improve the physical health of a user.

### SUMMARY

In accordance with embodiments herein, a correction system is provided that includes a sensor system comprising a sensor configured to obtain sensor data related to a position of a body part of a user in relation to an electronic device. The electronic device can include one or more processors and a data storage device having executable instructions accessible by the one or more processors. Responsive to execution of the instructions, the one or more processors are configured to determine a position of the body part of the user based on the sensor data obtained from the sensor and determine whether the position of the body part is at a determined position related to ensuring physical health of the body part. The one or more processors are also configured to alert the user to vary the position of the body part when determining the body part is not at the determined position related to ensuring physical health of the body part.

Optionally, the sensor system also includes a magnet configured to be detected by the sensor. In one aspect, the magnet may be coupled to a wearable device configured to be worn by the user. In another aspect, the wearable device can be at least one of glasses, headband, headphones, necklace, earring, bracelet, ring, or cufflink. Alternatively, the sensor may be coupled to at least one of a wearable device or a wrist support retractably coupled within the electronic device.

In one example, to determine whether the position of the body part is at the determined position related to ensuring physical health of the body part can include analyzing, utilizing artificial intelligence, body part positions of other users and determining the determined position based on the body part positions of the other users. In another example, to determine whether the position of the body part is at the determined position related to ensuring physical health of the body part may include obtaining user characteristics related to the body part and determining the determined position based on the user characteristics. In yet another example, the one or more processors can further be configured to determine whether the position of the body part is at the determined position at a first time; and update the determined position after receiving additional body part positions of additional other users at a second time that is later than the first time. Optionally, alerting the user to correct the position of the body part may include at least one of a popup on a display of the electronic device, an auditory message from a speaker of the electronic device, haptic feedback from a component of the electronic device, or haptic feedback from a wearable device that includes the sensor. In one aspect the electronic device can further comprise a retractable wrist support configured to extend from the electronic device. In another aspect, the one or more processors may be further configured to automatically extend the retractable wrist support based on at least one of the sensor data or a user characteristic.

In accordance with embodiments herein a correction system is provided that can include a first wearable device configured to be worn by a user of an electronic device, where the first wearable device may include a magnet. The system can also include a second wearable device configured to be worn by the user of the electronic device, where the second wearable device may include a sensor configured to obtain sensor data based on detecting the magnet. The electronic device can include one or more processors and a data storage device having executable instructions accessible by the one or more processors. Responsive to execution of the instructions, the one or more processors may be configured to obtain, from the sensor, the sensor data related to the body part of the user of the electronic device and determine a position of the body part of the user based on the sensor data obtained from the sensor. The one or more processors may also be configured to determine whether the position of the body part can be at a determined position related to ensuring physical health of the body part and alert the user to vary the position of the body part when determining the body part is not at the determined position related to ensuring physical health of the body part.

Optionally, the first wearable device can be an earring and the second wearable device may be a necklace and the one or more processors can be configured to determine the position of a head of the user based on the sensor data. In one aspect, the first wearable device may be a ring and the second wearable device can be a bracelet and the one or more processors may be configured to determine the position of the wrist of the user based on the sensor data. In another aspect, to determine whether the position of the body part is at the determined position related to ensuring physical health of the body part can include analyzing, utilizing artificial intelligence, body part positions of other users and determining the determined position based on the body part positions of the other users. In one example, alerting the user to correct the position of the body part may include at least one of a popup on a display of the electronic device, an auditory message from a speaker of the electronic device, haptic feedback from a component of the electronic device, or haptic feedback from a wearable device that includes the sensor. In another example, the electronic device may also include a retractable wrist support configured to extend from the electronic device. In yet another example, the one or more processors may be further configured to automatically extend the retractable wrist support based on at least one of the sensor data or a user characteristic.

In accordance with embodiments herein, a computer program product is provided that can include a non-transitory computer readable storage medium comprising computer executable code. The computer executable code is configured to determine a position of a body part of the user in relation to an electronic device based on sensor data obtained from a sensor, and determine whether the position of the body part may be at a determined position related to ensuring physical health of the body part, and alert the user to vary the position of the body part when determining the body part is not at the determined position related to ensuring physical health of the body part. Optionally, to determine whether the position of the body part is at the determined position related to ensuring physical health of the body part can include analyzing, utilizing artificial intelligence, body part positions of other users and determining the determined position based on the body part positions of the other users.

### BRIEF DESCRIPTION OF THE DRAWINGS

FIG. 1 illustrates a perspective view of correction system formed in accordance with an embodiment described herein.

FIG. 2 illustrates a perspective view of a sensor system formed in accordance with an embodiment described herein.

FIG. 3 illustrates a perspective view of a sensor system formed in accordance with an embodiment described herein.

FIG. 4 illustrates a perspective view of an electronic device formed in accordance with an embodiment described herein.

FIG. 5 illustrates a schematic block diagram of a control system for an electronic device in accordance with an embodiment described herein.

FIG. 6 illustrates a block flow diagram of a method of correcting body part positioning in accordance with an embodiment described herein.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments as claimed but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment,” “in an embodiment” or the like in various places throughout this specification are not necessarily all referring to the same embodiment.

Furthermore, the described features, structures or characteristics may be combined in any suitable manner in one or more embodiments. In the following description, numerous specific details are provided to give a thorough understanding of the various embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The term “base subassembly” refers to any and all assemblies of an electronic device that do not contain a display. The base subassembly may include an input device such as keys of a keyboard, ports such as USB ports, activation buttons, touch pads, or the like. The base subassembly may include a housing for one or more processors, a storage device, functional circuitry, etc. The base subassembly may be referred to as C/D cover with the C side being the side typically having the keyboard, activation buttons, touch pad, etc., and the D side being opposite of the C side, and typically having legs, pads, etc. for sitting the electronic device on a surface.

The term “display subassembly” refers to any and all assemblies of an electronic device that contain a display. The display subassembly may include a screen, touch screen, camera, bevel, etc. The display subassembly may couple to a base subassembly, including via hinge such that the display subassembly and base subassembly can pivot about a pivot axis. The display subassembly may be detachable from the base subassembly. The display subassembly may also not include a base subassembly, such as a tablet, smartphone, or the like. Instead, all input devices, ports, etc. are part of the display subassembly. The display subassembly may be referred to as an A/B cover with the A side typically being the side of the display subassembly that does not contain a display, and opposite the B side that does include the display.

The term “user characteristics” refers to any and all traits, parameters, details, information, data, etc. related to a user of a device. User characteristics include characteristics that are input by a user or other, including profile information, user settings, user passwords, application settings, etc. User characteristics also include characteristics obtained by the electronic device, including electronically through communications, and electronically through sensors. Specifically, user characteristics may be obtained through a network, cloud, server, remote device, etc. User characteristics may also be obtained from a disk, thumb drive, hard drive, memory stick, remote device from a wired connection, remote device from a wireless connection, or the like. User characteristics may also be obtained from sensors, including cameras, microphones, motion sensors, fingerprint scanners, biosensors, etc. User characteristics can include age of user, permission settings of user, identification of user, inputs of user, etc.

The term “obtains” and “obtaining”, as used in connection with data, signals, information and the like, include at least one of i) accessing memory of an external device or remote server where the data, signals, information, etc. are stored, ii) receiving the data, signals, information, etc. over a wireless communications link between the base device and a secondary device, and/or iii) receiving the data, signals, information, etc. at a remote server over a network connection. The obtaining operation, when from the perspective of a base device, may include sensing new signals in real time, and/or accessing memory to read stored data, signals, information, etc. from memory within the base device. The obtaining operation, when from the perspective of a secondary device, includes receiving the data, signals, information, etc. at a transceiver of the secondary device where the data, signals, information, etc. are transmitted from a base device and/or a remote server. The obtaining operation may be from the perspective of a remote server, such as when receiving the data, signals, information, etc. at a network interface from a local external device and/or directly from a base device. The remote server may also obtain the data, signals, information, etc. from local memory and/or from other memory, such as within a cloud storage environment and/or from the memory of a personal computer.

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

Electronic devices are provided with an alignment systems that include wearable devices utilized to determine the location, alignment, positioning, etc. of a body part. The wearable devices can be earrings, necklaces, glasses, bracelets, rings, headbands, headphones, cufflinks, or the like that include sensors for detecting the position of a body part. For example, a bracelet and ring can be utilized to determine the position and movement of the wrist. Headphones, glasses, earrings, etc. can be used in combination with a necklace, cufflink, or the like to determine the position of the head of a user compared to their shoulders or back. Clothing with sensors can similarly be utilized. In response to detecting that a body part is not correctly aligned, a corrective alert can be provided to the user of the system. A corrective alert can include a voice command, audible noise, haptic vibration or feedback, intermittent lighting or flashing, or the like to alert the user that their body part is not in a correct location. For example, a person may be alerted to sit up straight, tilt their head back, move their hand or wrist, etc. to provide a corrective action the user can implement.

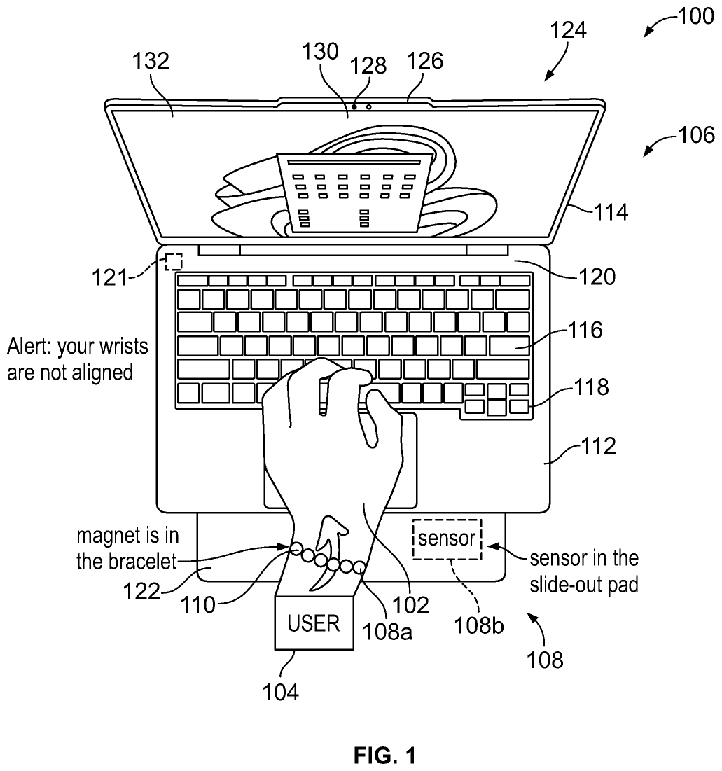

FIG. 1 illustrates a correction system 100 for correcting the positioning of a body part 102 of a user 104 of an electronic device 106. The body part 102 can include a head, neck, shoulders, wrist, hands, or the like of the user. The electronic device 106 can be a laptop computer, desktop computer, smart phone, personal digital assistant (PDA), tablet, iPad, iPhone, or the like. The electronic device 106 can be in communication with at least one sensor system 108 that may be associated with the user 104 and can be configured to detect and provide information that can be utilized to determine the position of a body part 102 of the user.

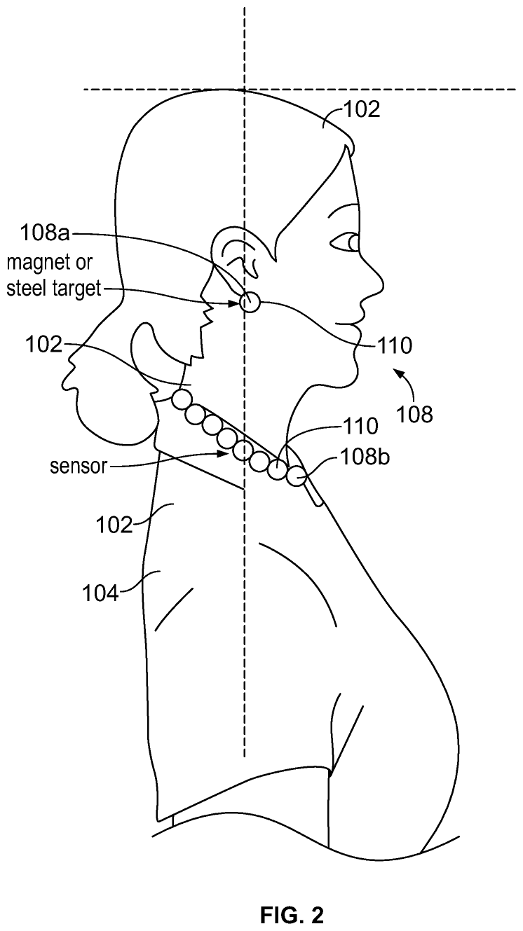

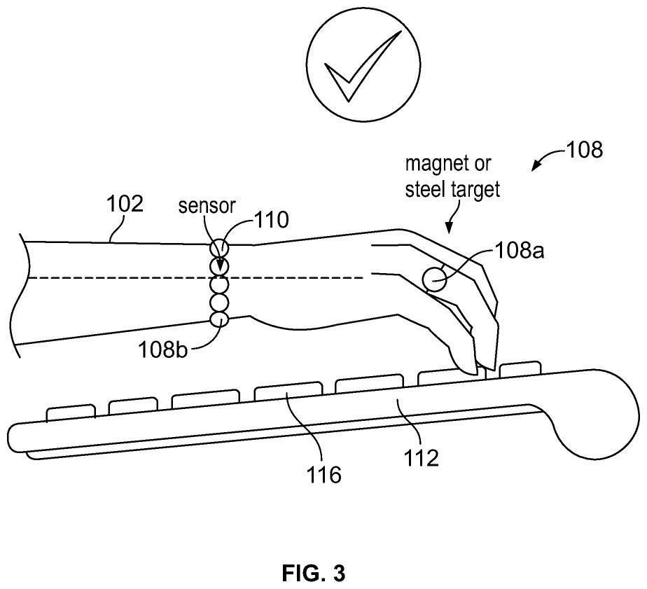

The sensor system 108 in one example can include both a magnet 108a and a sensor 108b configured to detect the magnet 108a. In one example the sensor 108b may be a Hall Effect sensor that is configured to detect the position of the magnet 108a with respect to the sensor 108b. In addition, in one embodiment the components (e.g. magnet 108a and sensor 108b) of the sensor system 108 can be coupled with or part of a wearable device 110. The wearable device 110 can be an earring, necklace, bracelet, glasses, article of clothing, cufflink, or the like. FIG. 2 illustrates an example of the wearable devices 110, where an earring can include a magnet 108a, and a necklace includes a sensor 108b and the magnet 108a and sensor 108b are used in combination to determine when a head (e.g. body part 102) of a user 104 is not positioned properly. While in this example a combination of a magnet 108a in an earring and a sensor 108b in a necklace is provided, alternatively, the magnet 108a could be in the necklace and the sensor 108b could be in the earring. In yet another example as illustrated in FIG. 3, a magnet 108a could be in a ring while a sensor 108b is in a bracelet, or vice versa, to determine the position of a wrist (e.g., body part 102) of a user. In other examples a glasses and necklace combination, cufflink and ring combination, or the like can be used as the magnet 108a and sensor 108b combination to make determinations related to the positioning of a body part 102 of a user 104.

With reference back to FIG. 1, the electronic device 106 in one example can include a base subassembly 112 and a display subassembly 114. In one example, the base subassembly 112 and the display subassembly 114 can rotatably couple to one another. In this manner, for transportation the base subassembly 112 and the display subassembly 114 can be folded together to compactly engage one another.

The base subassembly 112 can include a keyboard 116 for typing and inputting information. The base subassembly 112 can also optionally include a base speaker 118 configured to provide auditory sounds, messages, instructions, etc. The base subassembly 112 can also include a base microphone 120 for detecting auditory sounds, including instructions from the user 104. In addition, the base subassembly 112 can include one or more additional sensors 121 for interacting with, communicating with, etc. a remote sensor 108b, including those coupled to or with a wearable device 110.

In one embodiment the base subassembly 112 can also include a retractable wrist support 122 that can be disposed within a compartment of the base subassembly 112 in a first position and be extended from the base subassembly 112 in a second position. The retractable wrist support can be a board, include padding, or the like that can be used by the user 104 to rest their wrists on during use of the keyboard 116 of the base subassembly. In one example embodiment as provided in FIG. 1, the wrist support can be part of the sensor system 108. In particular, the user can have a wearable device 110 such as a bracelet that includes a magnet 108a while the wrist support 122 can include a sensor 108b that detects the position of the wrist in relation to the wrist support 122. If the wrist is determined to be in an incorrect position an alert such as a communication can be provided to adjust the positioning of the wrist to a correct position.

The display subassembly 114 can include one or more displays 124 or screen coupled to one another for presentation on information. In one example the display 124 may be a touch screen. The display subassembly 114 can also include a display speaker 126, display microphone 128, and a display camera 130 for obtaining images, including for obtaining user characteristics related to the user 104. The display subassembly 114 may also include additional sensors 132, magnets, or the like for interacting, communicating, etc. with one or more sensors 108b remote from the display subassembly. These remote sensors 108b can include the sensors 108b coupled to or with one or more wearable devices 110. In one example, the remote sensors 108b can be Hall Effect sensors configured to provide the position of a body part 102 of the user 104.

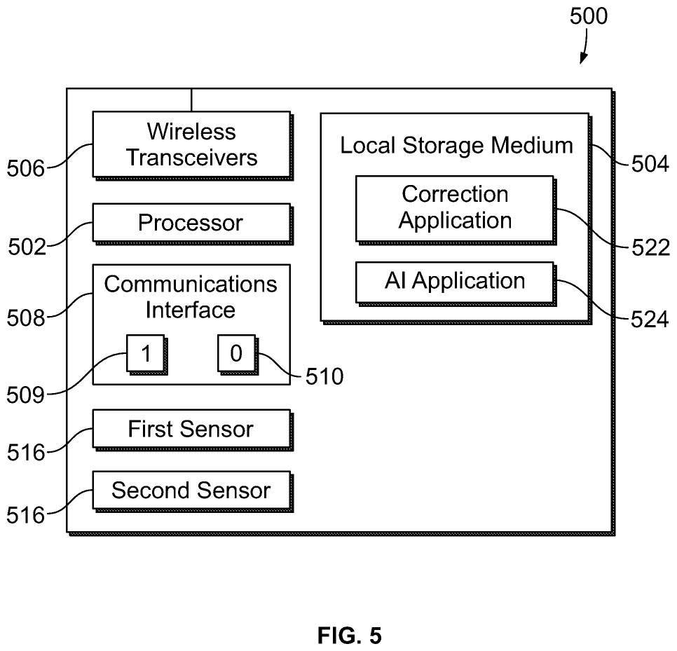

The electronic device 106 can also include one or more processors, a storage device, transceiver, or the like (see FIG. 5). The processors, storage device, transceiver, etc. can be utilized with the sensor system 108 to determine the position of a body part 102. In addition, the memory can include a correction application (FIG. 5) that includes instructions to determine whether the position of a body part 102 is at a correct, or determined, location, and then provide an alert that the body part 102 needs to be moved. In an example, positioning data received from the sensor system 108 can be compared to determined positioning data. In one example, the determined positioning data can be determined from analyzing positioning data from numerous other individuals that have utilized similar sensor systems to the sensor system being utilized by user 104. In another example, the determined positioning data can be based on a calculation, model, or the like.

In yet another example, the user 104 can have a profile where the user input information about themselves including height, weight, or other user characteristics and the determined positioning data is determined from the input data. In yet another example, while making a profile the correction application can obtain user characteristics, including through use of an additional sensor 121, 132 such as a camera of the electronic device. The correction application can then use the obtained user characteristics to determine the determined positioning data. In one example, artificial intelligence (AI) can be utilized based on sets of data related to user characteristics of the user, and numerous other users to make determinations regarding the correctness of the position of the body part 102. In particular, different users have different heights, weights, body part size, body part shape, etc. that can result in different positioning for the best positioning of the body part 102. By utilizing AI, the determined positioning data can be repeatedly updated over time to improve the determined positioning data, and thus positioning of the body part 102 of the user 104.

Once the correction application (FIG. 5) determines correction in positioning of a body part 102 is needed, an alert can be provided. The alert can be a message displayed on a display 124, an auditory sound, beep, or otherwise, an auditory warning, instruction, suggested correction, or the like to prompt the user 104 to place their body part 102 in a correct location, or the like.

In one example, if the head of a user is slouched, the correction applicant can display to outline can show the user 104 where their head should be located. In another example, the correction application can vary the keyboard 116, a display 124, or other component of the electronic device 106 attempt to facilitate correction of the incorrect location of the body part. For example, the correction application can instruct and cause the automatic mechanical movement of a display 124 to tilt a screen upwardly so a user 104 does not have to slouch to get a clear view of the screen. Similarly, the retractable wrist support 122 can be automatically extended, the keyboard automatically and mechanically tilted or moved, or the like to facilitate the correction of the body part 102 of the user 104. In this manner, not only can the system detect and determine that a body part 102 is in an incorrect position, but additionally provide correction actions for correcting the location of the body part 102.

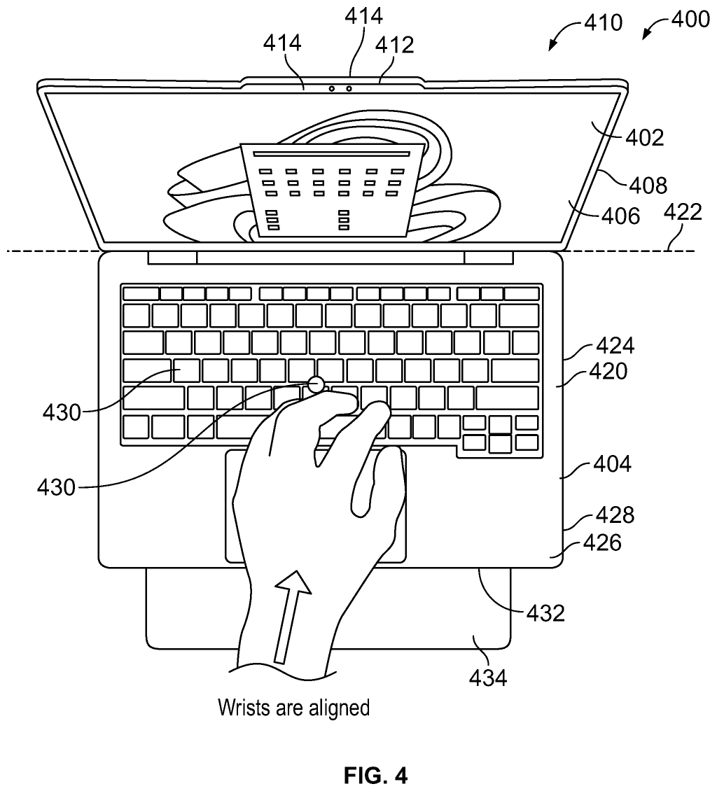

FIG. 4 illustrates a perspective view of an electronic device 400 formed in accordance with an embodiment described herein. The electronic device 400 includes a display subassembly 402 and a base subassembly 404 that are pivotably coupled with each other. The electronic apparatus in example embodiments is a laptop computer, desktop computer, smart phone, personal digital assistant (PDA), tablet, iPad, iPhone, or the like. The display subassembly 402 in one example is a tablet computer or other electronic device that may be detached from the base subassembly 404.

The display subassembly includes a housing 406 with an outer shell 408 and a display 410. The display 410 may be a touchscreen or non-touch sensitive display. The display 410 visually presents information to a user of the electronic device 400 or the display subassembly 402. In one embodiment, adjacent to the display 410 on the first side is a bezel 412 that can include one or more electronic device sensors 414 such as a camera, motion detector, infrared camera, microphone, or the like. The electronic device sensors 414 may be utilized to obtain user characteristics that are used by one or more processor to determine an identification of the individual using the electronic device. For example, a camera may be utilized along with photo identification software to identify a person in from the display. In another example, a microphone with voice recognition software can be used to identify the user. The camera and/or microphone can also be utilized to obtain user characteristics that include the number of individuals viewing the display at one time. While illustrated as having a single display 410 that is coupled to the base subassembly 404, in other example embodiments the display subassembly 402 can be separate and/or detached from the base subassembly 404. In addition, the electronic device 400 can include multiple display subassemblies that each can be controlled by a base subassembly 404.

Disposed within the housing 406 of the display subassembly 402 are one or more processors, one or more storage devices, a transceiver, battery, circuitry, etc. Specifically, and components utilized to operate the electronic device 400 are placed within the housing 406 of the display subassembly.

The base subassembly 404 includes a housing 420 that couples with the housing 406 of the display subassembly 402. In one example, the display subassembly 402 and the base subassembly 404 are pivotably coupled with each other along a pivot axis 422. Rotation about the pivot axis 422 may occur as the result of an elongated hinge, plural hinges, rotational elements, detachable rotational elements, or the like.

The housing 424 forms at least part of the exterior surface of the base subassembly 404. The housing 424 has a first side 426 and an opposite second side 428. The first side 426 in one example is an input side that includes input devices 430 such as keyboards, touch sensitive surface, actuation buttons, etc. The first side can be considered the C side of the electronic device 400. Meanwhile, the second side 428 is a bottom side of the electronic device that engages a surface when the electronic device is in use. To this end, the second side may have pads, feet, pads, vents, etc. The second side 428 can be considered the D side of the electronic device 400.

In one embodiment, the base subassembly 404 also includes one or more processors, circuitry, etc. in the housing 424. These processors may be similar or identical to the processors in the display subassembly 402. Alternatively, the processors may have more processing capability (e.g., power) relative to the processors. The processors of the display subassembly 402 and/or the processors of the base subassembly 404 may receive signals from the input devices 430 and/or the display 410 and can perform operations based on or use these signals, such as by controlling the information presented on the display 410.

In one embodiment, the housing 424 of the base subassembly 404 defines at least one auxiliary compartment 432 of size and shape to receive a wrist support 434. The wrist support 434 in one example may be an elongated padded body. To this end, in one example, the auxiliary compartment 432 can contain the wrist support 434 and include a mechanical arm that can extend and retract the wrist support 434. In one example, the mechanical arm can be spring loaded such that when the wrist support is pushed inward, the wrist support then unlatches and extends away from the auxiliary compartment. Alternatively, the wrist support 434 can include rails, rack and carriage, or the like for extending and retracting the wrist support. In one example the wrist support 434 can include at least one sensor that can be configured to detect the movement and position of a magnet.

FIG. 5 illustrates a simplified block diagram of a control system 500 for an electronic device. In one example, the electronic device may be any of the electronic devices as described in relation to FIGS. 1-4. To this end, the components of the electronic device may be located only in a display subassembly, only in a base subassembly, or may be located in both the display subassembly and display subassembly.

The control system 500 includes components such as one or more processors 502 (e.g., a microprocessor, microcomputer, application-specific integrated circuit, etc.), one or more local storage medium (also referred to as a memory portion) 504, one or more wireless transceivers 506, a user interface 508 which includes one or more input devices 509 and one or more output devices 510, a power module, a component interface, and one or more sensors 516. All of these components can be operatively coupled to one another, and can be in communication with one another, by way of one or more internal communication links, such as an internal bus.

The local storage medium 504 can encompass one or more memory devices of any of a variety of forms (e.g., read only memory, random access memory, static random-access memory, dynamic random-access memory, etc.) and can be used by the processor 502 to store and retrieve data. The data that is stored by the local storage medium 504 can include, but need not be limited to, operating systems, applications, obtained context data, and informational data. Each operating system includes executable code that controls basic functions of the device, such as interaction among the various components, communication with external devices via the wireless transceivers 506 and/or the component interface 514, and storage and retrieval of applications and context data to and from the local storage medium 506.

The input and output devices 509, 510 may each include a variety of visual, audio, and/or mechanical devices. For example, the input devices 509 can include a visual input device such as an optical sensor or camera, an audio input device such as a microphone, and a mechanical input device such as a keyboard, keypad, selection hard and/or soft buttons, switch, touchpad, touch screen, icons on a touch screen, a touch sensitive areas on a touch sensitive screen and/or any combination thereof. Similarly, the output devices 510 can include a visual output device such as a liquid crystal display screen, one or more light emitting diode indicators, a mechanical output device such as a vibrating mechanism, etc. The display may be touch sensitive to various types of touch and gestures. As further examples, the output device(s) 510 may include a touch sensitive screen, a non-touch sensitive screen, a text-only display, a smart phone display, and/or any combination thereof.

The user interface 508 permits the user to select one or more of a switch, button, or icon to collect context data, and/or enter context data. The user interface 508 can also direct the one or more sensors 516 to obtain user characteristics. As an example, in one embodiment a sensor is a camera that can take a photo or video (e.g., capture image data). Alternatively, the sensor can be a microphone, motion sensor, global positioning sensor, or the like. Each sensor may provide user characteristics that may be utilized by the one or more processors to determine a user of the electronic device along with current electronic device characteristics such as the angle of a display in relation to the user.

The local storage medium 506 in one embodiment stores various content including, but not limited to correction application 522. The correction application 522 includes executable code that utilizes an operating system to perform numerous functions related to correcting positioning of a body part of a user. Such functions can include obtaining user characteristics to determine an identity of the user, and to determine whether a user has a user profile. The correction application 522 can also prompt a user to receive inputs from the user related to a user profile, related to the posture or body part location of a user, or the like.

The correction application 522 can also obtain sensor information from sensors and utilize the sensor information to determine the location and/or positioning of a body part of the user. In one example, the sensor can be a Hall Effect sensor that can detect movement and a location of a magnet associated with the user. In an example the magnet can be on, within, associated with, etc. a wearable device such as a headband, headphones, visors, glasses, earrings, necklaces, bracelets, rings, clothing, other jewelry, or the like.

The correction application 522 can also determine the position of a body part of the user based on the sensor information. The correction application can then determine whether the position of the body part is the most physically healthy position of the user. Such determination may be made by comparing the determined position of a body part compared to a determined position of the body part. In one example, the determined position can be a position recorded, or saved, within the local storage medium as part of a user profile. In another example, the determined position can be calculated based on user characteristics input into the correction application such as height, weight, or the like as a result of prompts from the correction application 522. In one example such prompts occur when creating a user profile. In another example the determined position is determined by the correction application by utilizing numerous other measurement, calculations, or the like of individuals sitting at or using the electronic device. Such other individuals can be real individuals that similarly have a correction application on an electronic device that communicates with the correction application. Alternatively, the individuals can be a set of real individuals that are measured, observed, etc. and inputted into the correction application. In yet another alternative embodiment, the other individuals can include one or more digital or composite individuals created by an electronic device or program using data, simulations, or the like. Such determinations for each and other examples can include utilizing mathematical equations, algorithms, mathematical models, or the like.

The correction application 522 can also be configured to determine whether to alert or communicate to a user that their body part is incorrectly positioned. In one example, if the body part is not in the determined position, a determination is made to provide an alert or communication. In another example, the threshold is provided related to the determined position. For example, the threshold can be a distance measurement such as a determined number of inches, centimeters, millimeters, etc. a body part is from the determined position. In another example the threshold can be an angle measurement where a number of degrees from a determined angle represents the threshold. In yet another example a combination of distance or angle and time can represent the threshold. So if a user's head is off center by more than ten (10) degrees for at least five (5) consecutive seconds, a determination can be made to alert the user. In other examples the threshold can be a percentage such as a percent variance from a determined position. By using thresholds before alerting a user of desired correction, annoyance as a result of constant corrections can be avoided.

Additionally, the correction application 522 can be configured to determine and provide the alert, communication, etc. to let the user know correction of the position of the body part is needed. For example, the alert can be provided in a profile or predetermined by a user as provided in settings. The correction application 522 can then select the method chosen by the user. Alternatively, the alert may be adjusted or varied based on the amount of correction needed. So, if a user has their wrist positioned two (2) degrees off during typing, an initial alert such as haptic feedback of a wrist support can be provided. Whereas if a more severe angle such as five (5) degrees is detected an auditory command request change may be provided. In an alternative embodiment, the alert itself can vary. For example, the further away an individual gets from the determined position the more intense haptic feedback can be provided. Alternatively a sound, light, etc. can grow in intensity the further away a user becomes from the determined position. In one example the correction application can utilize numerous different corrections (e.g., auditory, haptic, visual, instructions, popup, etc.) and make determinations related to which causes the user to best take the corrective action. So, if haptic feedback is provided and it takes fifteen (15) seconds to correct the position of the body part, and a next time an auditory message is provided and it only takes five (5) second to correct the position, the correction application 522 will use the auditory command for that user.

The correction application 522 in another example can provide instructions to adjust or vary components of the electronic device to facilitate correction for a user. For example, a wrist support can automatically extend from electronic device when the user is not correctly positioning their wrists. In another example, a screen can tilt to accommodate the position of a user's head for viewing information on a display.

The local storage medium 506 can also include an AI application 524. In one example the AI application operates in unison with the correction application to make determinations related to the current position of a body part of the user, whether the current position determined is the most physically healthy position for the user, what correction, alert, communication, or otherwise the electronic device should provide the user based on the determination of whether the user has a body part in the most physically healthy position for the user, or the like. By utilizing the AI application, the determination and corrective actions can be tailored for the user, adapting to the user size, shape, form, etc.

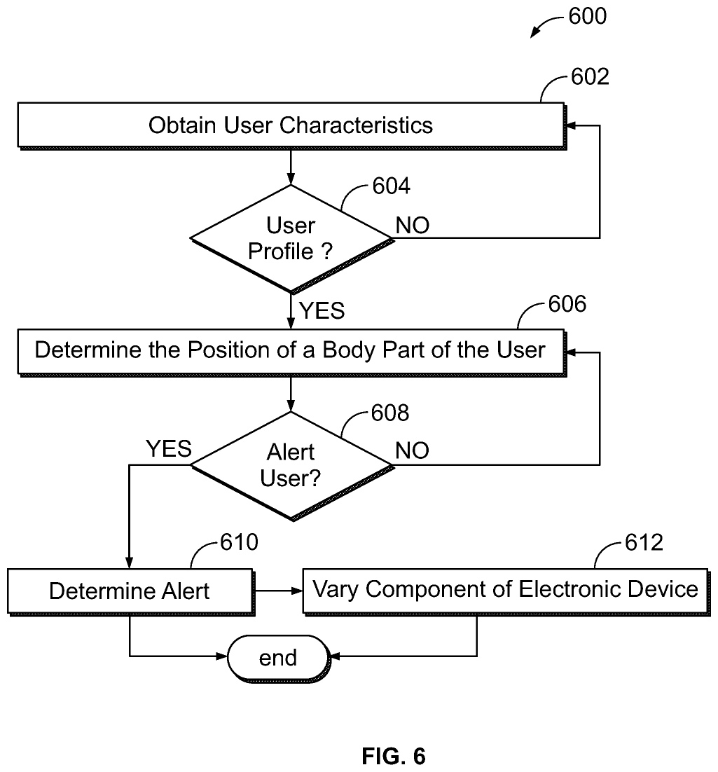

FIG. 6 illustrates a block flow diagram of a method 600 of correcting positioning of a body part of a user of an electronic device. In one example, an electronic device of any of the FIGS. 1-4 and/or control system of FIG. 5 can be utilized to implement the method 600. In another example, the control system of FIG. 5 is used to execute instructions to provide the method 600. The electronic device may include a personal computer, laptop computer, tablet, phone, watch, smart device, iPad, iPod, or the like.

At 602, the one or more processors obtain user characteristics. In one example, the user characteristics are obtained via detecting an individual using the electronic device using a sensor such a camera, microphone, motion detector, or the like. To this end, facial recognition, voice recognition, artificial intelligence (AI) modeling, etc. may be used to obtain the user characteristics. In addition, information input into the electronic device may be used to obtain the user characteristic, including the identity of the individual using the electronic device. Such input information includes login information, passwords, application logins and passwords, permission settings, fingerprint scanning, retinal scanning, user profile, user settings, or the like. User characteristics can include the age of a user, permission settings of a user, identification of a user, inputs of a user, or the like. In each instance, the user characteristic obtained provides information related to the identity of the individual using the electronic device.

At 604, the one or more processors determines if a user profile and/or settings are available for the individual using the electronic device and/or the application being used. In particular, based on both user characteristics obtained, and electronic device characteristics obtained, determinations are made regarding the identity of the individual using the electronic device along with applications being utilized by the electronic device. Such determinations may be made using a mathematical model, lookup table, decision tree, algorithm, mathematical function, or the like. If at 604, a determination cannot be made of the availability of a user profile or setting information, then the one or more processors continue to obtain user characteristics and electronic device characteristics.

If at 604, the one or more processors are able to obtain a user profile and/or settings, then at 606, the one or more processors determine the position of a body part of the user. The body part can be a head, neck, back, shoulder, elbow, wrist, hand, or the like. In one example to determine the position of the body part sensor data from a sensor system is received. In an example the sensor system can include a combination of a magnet and a sensor both associated with the body part of the user. In one example the magnet can be in a wearable device such as headphones while the sensor is in a wearable device that is a necklace. The sensor system can then determine the position and location of the head of the individual. Alternatively the detector can be in a wrist support and the magnet in a wearable device such as a ring. In this example a determination can be made regarding the position of the user's wrist when using a keyboard. In each example, the position, angle, tilt, location, etc. of the body part may be continuously and repeatedly determined in real-time.

At 608, the one or more processors determine whether to alert the user to correct the position of the body part. The one or more processors determine a determined position that the body part is expected to be based on any of the methods previously described herein. Then, the processors determine when to alert the user to correct the position of the body part. In examples a threshold measurement, angle, time period, or the like must be reached before the alert is provided as described in detail above. If a determination is made not to alert the user, the one or more processors continue obtaining and analyzing sensor data to determine if an alert is to be provided.

If at 608 a determination is made that an alert is needed, then at 610 a determination is made regarding the type of alert. The alert can be a popup on a display, auditory instruction, sound, beep, haptic feedback, a light indicator, or the like.

Optionally, at 612, the one or more processors can vary or adjust a component of the electronic device based on the position of the body part. In one example a display screen can be tilted at an angle to facilitate the correct posture of a user. In another example a wrist support within an internal compartment of the electronic device can mechanically and automatically extend from the electronic device for use.

As will be appreciated by one skilled in the art, various aspects may be embodied as a system, method, or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random-access memory (RAM), a dynamic random-access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code embodied on a storage medium may be transmitted using any appropriate medium, including but not limited to, wireless, wireline, optical fiber cable, RF, et cetera, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. These program instructions may be provided to a processor of a general-purpose computer, special purpose computer, or other programmable data processing device or information handling device to produce a machine, such that the instructions, which execute via a processor of the device implement the functions/acts specified.

The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

Although illustrative example embodiments have been described herein with reference to the accompanying figures, it is to be understood that this description is not limiting, and that various other changes and modifications may be affected therein by one skilled in the art without departing from the scope or spirit of the disclosure.

The modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only and are thus not intended to limit in any way the definition and/or meaning of the term “controller.” The modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A correction system comprising:
a sensor system comprising a sensor configured to obtain sensor data related to a position of a body part of a user in relation to an electronic device; and
the electronic device including one or more processors and a data storage device having executable instructions accessible by the one or more processors;
wherein, responsive to execution of the instructions, the one or more processors are configured to:
determine a position of the body part of the user based on the sensor data obtained from the sensor;
determine whether the position of the body part is at a determined position related to ensuring physical health of the body part; and
alert the user to vary the position of the body part when determining the body part is not at the determined position related to ensuring physical health of the body part.

2. The correction system of claim 1, wherein the sensor system further comprises a magnet configured to be detected by the sensor.

3. The correction system of claim 2, wherein the magnet is coupled to a wearable device configured to be worn by the user.

4. The correction system of claim 3, wherein the wearable device is at least one of glasses, headband, headphones, necklace, earring, bracelet, ring, or cufflink.

5. The correction system of claim 3, wherein the sensor is coupled to at least one of a wearable device or a wrist support retractably coupled within the electronic device.

6. The correction system of claim 1, wherein to determine whether the position of the body part is at the determined position related to ensuring physical health of the body part includes analyzing, utilizing artificial intelligence, body part positions of other users and determining the determined position based on the body part positions of the other users.

7. The correction system of claim 6, wherein to determine whether the position of the body part is at the determined position related to ensuring physical health of the body part includes obtaining user characteristics related to the body part and determining the determined position based on the user characteristics.

8. The correction system device of claim 6, the one or more processors further configured to determine whether the position of the body part is at the determined position at a first time; and update the determined position after receiving additional body part positions of additional other users at a second time that is later than the first time.

9. The correction system of claim 1, wherein alerting the user to correct the position of the body part includes at least one of a popup on a display of the electronic device, an auditory message from a speaker of the electronic device, haptic feedback from a component of the electronic device, or haptic feedback from a wearable device that includes the sensor.

10. The correction system of claim 1, wherein the electronic device further comprises a retractable wrist support configured to extend from the electronic device.

11. The correction system of claim 10, wherein the one or more processors are further configured to automatically extend the retractable wrist support based on at least one of the sensor data or a user characteristic.

12. A correction system, comprising:
a first wearable device configured to be worn by a user of an electronic device, the first wearable device including a magnet;
a second wearable device configured to be worn by the user of the electronic device, the second wearable device including a sensor configured to obtain sensor data based on detecting the magnet;
the electronic device including one or more processors and a data storage device having executable instructions accessible by the one or more processors;
wherein, responsive to execution of the instructions, the one or more processors are configured to:
obtain, from the sensor, the sensor data related to the body part of the user of the electronic device;
determine a position of the body part of the user based on the sensor data obtained from the sensor;
determine whether the position of the body part is at a determined position related to ensuring physical health of the body part; and
alert the user to vary the position of the body part when determining the body part is not at the determined position related to ensuring physical health of the body part.

13. The correction system of claim 12, wherein the first wearable device is an earring and the second wearable device is a necklace and the one or more processors are configured to determine the position of a head of the user based on the sensor data.

14. The correction system of claim 12, wherein the first wearable device is a ring, and the second wearable device is a bracelet, and the one or more processors are configured to determine the position of the wrist of the user based on the sensor data.

15. The correction system of claim 12, wherein to determine whether the position of the body part is at the determined position related to ensuring physical health of the body part includes analyzing, utilizing artificial intelligence, body part positions of other users and determining the determined position based on the body part positions of the other users.

16. The correction system of claim 12, wherein to alert the user to correct the position of the body part includes at least one of a popup on a display of the electronic device, an auditory message from a speaker of the electronic device, haptic feedback from a component of the electronic device, or haptic feedback from a wearable device that includes the sensor.

17. The correction system of claim 12, wherein the electronic device further comprises a retractable wrist support configured to extend from the electronic device.

18. The correction system of claim 17, wherein the one or more processors are further configured to automatically extend the retractable wrist support based on at least one of the sensor data or a user characteristic.

19. A computer program product comprising a non-transitory computer readable storage medium comprising computer executable code to:
determine a position of a body part of the user in relation to an electronic device based on sensor data obtained from a sensor;
determine whether the position of the body part is at a determined position related to ensuring physical health of the body part; and
alert the user to vary the position of the body part when determining the body part is not at the determined position related to ensuring physical health of the body part.

20. The computer program product of claim 19, wherein to determine whether the position of the body part is at the determined position related to ensuring physical health of the body part includes analyzing, utilizing artificial intelligence, body part positions of other users and determining the determined position based on the body part positions of the other users.
